(19) United States
(12) Patent Application Publication
(10) Pub. No.: US 2009/0157161 A1
(43) Pub. Date: Jun. 18, 2009

(54) Percutaneous Nitinol Stent Extraction Device

(75) Inventors: Pranav Desai, Anaheim Hills, CA (US); Rajesh Khanna, Norrisdale (CA)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/252,251

(22) Filed: Oct. 15, 2008

Related U.S. Application Data
(60) Provisional application No. 60/982,385, filed on Oct. 24, 2007.

(51) Int. Cl. A61F 2/06; A61M 25/00

(52) U.S. Cl. 623/1.11; 604/264

(57) ABSTRACT

A minimally invasive catheter system and method for extraction of a shape memory device such as a nitinol stent from inside a tubular organ, is provided. The catheter system comprises a multi-lumen tube with at least one expandable balloon and an extraction device. The multi-lumen tube has multiple ports, which are used for injecting fluid inside the tubular organ and the expandable balloon, and inserting the extraction device. The catheter system is inserted inside the lumen of the tubular organ percutaneously. A cold fluid is injected into the expandable balloon and the lumen of the tubular organ. This cold fluid converts the shape memory device from an expanded state to a collapsed state. The shape memory device in the collapsed state is then removed with the help of the extraction device.

### RELATED APPLICATION

This application claims priority to U.S. Provisional Application No. 60/982,385, filed Oct. 24, 2007, the entire disclosure of which is incorporated by reference herewith.

### BACKGROUND OF THE INVENTION

1. Field of the Invention

The present invention relates, in general, to medical devices, and more particularly, to minimally invasive systems and methods for the removal of medical devices from inside a body lumen, such as a blood vessel.

2. Background

Atherosclerosis is the deposition of fatty plaques on the luminal surface of arteries, which, in turn, causes narrowing of the cross-sectional area of the artery. Ultimately, this deposition blocks the blood flow distal to the lesion, causing ischemic damage to the tissues supplied by the artery. The narrowing of the coronary artery lumen causes destruction of heart muscle, resulting first in angina, followed by myocardial infarction, and finally death. Stents are often deployed in arteries, heart valves, and lumens of other tubular organs such as the biliary duct so as to ensure a smooth flow of blood or the body fluids through the arteries or the lumens. Stents are metal scaffolds that are permanently implanted in the diseased arterial segment to hold the lumen open and improve the blood flow. The placement of a stent in the affected arterial segment therefore prevents recoil and subsequent closing of the artery.

Stents are typically formed from malleable metals such as 300 series stainless steel, or from resilient metals such as super-elastic and shape memory alloys, e.g., Nitinol™ alloys, spring stainless steels, and the like. They can also, however, be formed from non-metal materials such as non-degradable or biodegradable polymers, or from bioresorbable materials such as levorotatory polylactic acid (L-PLA), polyglycolic acid (PGA), or other materials such as those described in U.S. Pat. No. 6,660,827.

A variety of stent geometries are known in the art including, without limitation, slotted tube-type stents, coiled wire stents, and helical stents. Stents are also classified into two general categories, based on their mode of deployment. The first type of stent is expandable upon application of a controlled force, such as the inflation of the balloon portion of a dilatation catheter, which, upon inflation of the balloon or other expansion methods, expands the compressed stent to a larger, fixed diameter, to be left in place within the artery at the target site. The second type of stent is a self-expanding stent formed from shape memory metal or super-elastic alloy such as nickel-titanium (NiTi) alloys that automatically expands or springs from a compressed state to an expanded shape that it remembers.

Exemplary stents are described in U.S. Pat. No. 4,553,545 to Maass et al.; U.S. Pat. Nos. 4,733,665 and 4,739,762 to Palmaz; U.S. Pat. Nos. 4,800,882 and 5,282,824 to Gianturco; U.S. Pat. Nos. 4,856,516, 4,913,141, 5,116,365 and 5,135,536 to Hillstead; U.S. Pat. Nos. 4,649,922, 4,886,062, 4,969,458 and 5,133,732 to Wiktor; U.S. Pat. No. 5,019,090 to Pinchuk; U.S. Pat. No. 5,102,417 to Palmaz and Schatz; U.S. Pat. No. 5,104,404 to Wolff; U.S. Pat. No. 5,161,547 to Tower; U.S. Pat. No. 5,383,892 to Cardon et al.; U.S. Pat. Nos. 5,449,373, 5,733,303, 5,843,120, 5,972,018, 6,443,982, and 6,461,381 to Israel et al.; U.S. Pat. Nos. 5,292,331, 5,674,278, 5,879,382 and 6,344,053 to Boneau et al.; U.S. Pat. Nos. 5,421,955, 5,514,154, 5,603,721, 5,728,158, and 5,735,893 to Lau; U.S. Pat. No. 5,810,872 to Kanesaka et al.; U.S. Pat. No. 5,925,061 to Ogi et al.; U.S. Pat. No. 5,800,456 to Maeda et al.; U.S. Pat. No. 6,117,165 to Becker; U.S. Pat. No. 6,358,274 to Thompson; U.S. Pat. No. 6,395,020 to Ley et al.; U.S. Pat. Nos. 6,042,597 and 6,488,703 to Kveen et al.; and U.S. Pat. No. 6,821,292 to Pazienza et al., which are all incorporated by reference herein.

Once a stent is deployed, in some cases, there is an unwanted growth of tissue around the stent. This tissue growth may block the blood flow in the tubular organ, thereby causing restenosis. Restenosis refers to the re-narrowing of an artery after the initially successful deployment of a stent. Further, in a high percentage of patients, the stent becomes the site of recurrent stenosis due to the thickening of the walls of an artery (neointimal proliferation). Moreover, in some cases, the stent is displaced from the site of deployment. In such cases, the stent needs to be replaced with another stent, removed or repositioned.

Stents can be removed either by open surgery or percutaneously. Percutaneous removal is minimally invasive. It causes less trauma to the patient, as compared to open surgery. Further, the recovery of the patients is faster. In addition, percutaneous removal can be performed in an out-patient setting. However, very few systems and methods exist for percutaneous removal of nitinol stents.

Hence, there remains a need for a minimally invasive system and method for percutaneous removal of a nitinol stent from inside a tubular organ.

### SUMMARY OF THE INVENTION

The present invention addresses the above problems by providing a minimally invasive catheter system for extracting a shape memory device from inside a tubular organ. The catheter system has a multi-lumen tube and an extraction device. The multi-lumen tube has at least one expandable balloon and multiple ports. The expandable balloon is inflated by infusing fluid into it through one of the ports. The temperature of the infused fluid converts the shape memory device from an expanded state to a collapsed state. The extraction device is inserted into the lumen of the tubular organ through one of the ports. The extraction device removes the collapsed shape memory device from inside the lumen of the tubular organ by pulling it into the catheter system.

More specifically, a minimally invasive catheter system for extracting a nitinol stent from inside a tubular organ is provided. The catheter system has a multi-lumen tube and an extraction device. The multi-lumen tube has at least one expandable balloon and multiple ports. The expandable balloon is inflated by infusing fluid into it through one of the ports. The temperature of the infused fluid converts the nitinol stent from an expanded state to a collapsed state. The extraction device, with a hook and a sheath, is inserted into the lumen of the tubular organ through one of the ports of the catheter. The hook dislodges the nitinol stent from the walls of the tubular organ and is used to move or retrieve the stent into the sheath. The dislodged nitinol stent is then transported out of the tubular organ with the help of the sheath.

According to one aspect of the invention, a method for percutaneous extraction of a shape memory device from inside a tubular organ, using a catheter system, is provided. The catheter system has a multi-lumen tube and an extraction device. The multi-lumen tube has at least one expandable balloon and multiple ports. The catheter system is inserted inside the lumen of the tubular organ till the catheter system reaches the point of placement of the shape memory device. Fluid is then infused into the expandable balloon through one of the lumens of the multi-lumen tube. The temperature of the infused fluid converts the shape memory device from an expanded state to a collapsed state. The collapsed shape memory device is then removed from inside the tubular organ by using the extraction device.

### BRIEF DESCRIPTION OF THE DRAWINGS

FIG. 1 illustrates a catheter system for extraction of a shape memory device from inside a tubular organ, according to one embodiment of the invention;

FIG. 2 illustrates a distal end of the catheter system with one expandable balloon, according to one embodiment of the invention;

FIG. 3 illustrates a proximal end of the catheter system, according to one embodiment of the invention;

FIG. 4 illustrates a distal end of the catheter system with two expandable balloons, according to one embodiment of the invention;

FIG. 5 is a flowchart illustrating the steps of percutaneous extraction of the shape memory device from inside the tubular organ, according to one embodiment of the invention;

FIG. 6 is a flowchart illustrating the steps of percutaneous extraction of the shape memory device from inside the tubular organ by using an expandable balloon, according to one embodiment of the invention;

FIG. 7 is a flowchart illustrating the steps of percutaneous extraction of the shape memory device from inside the tubular organ by using two expandable balloons, according to one embodiment of the invention;

FIGS. 8A, 8B, 8C, and 8D illustrate the stages of extraction of a shape memory device from inside a tubular organ, according to one embodiment of the invention.

### DETAILED DESCRIPTION OF THE PREFERRED EMBODIMENTS

The present invention is directed to a minimally invasive catheter system and associated method for percutaneous extraction of a shape memory device from inside a tubular organ. More specifically, it is directed to a system and method for removal of self-expanding stents from inside a lumen of a tubular organ such as a blood vessel. Self-expanding stents have shape memory. Self-expanding stents, hereinafter referred to as stents, are removed from inside the tubular organ by using the shape memory property. A stent deployed inside the tabular organ is converted from an expanded state to a collapsed state by cooling the stent. The stent in the collapsed state is then dislodged from the walls of the tubular organ and removed from inside the tubular organ.

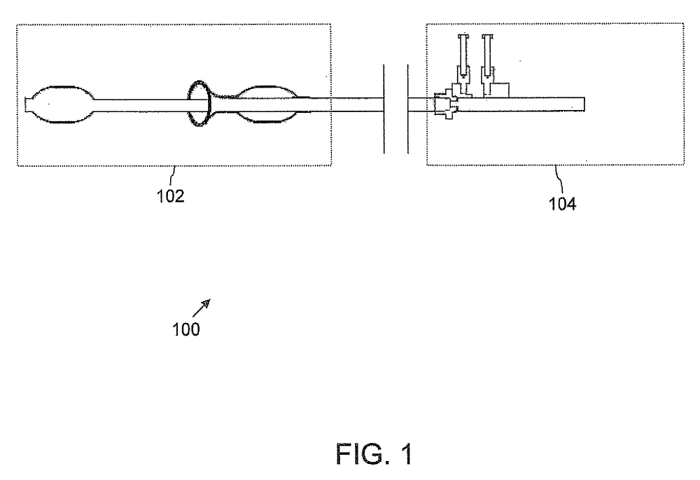

The catheter system includes a multi-lumen tube with at least one expandable balloon and an extraction device. The extraction device has a hook and a sheath. The catheter system is inserted into the lumen of the tubular organ, and the expandable balloon is inflated by infusing fluid. The temperature of the infused fluid converts the shape memory device from an expanded state to a collapsed state. The hook dislodges the collapsed shape memory device from the walls of the tubular organ and moves or pulls the collapsed shape memory device into a sheath. The sheath transports the collapsed shape memory device out of the tubular organ. The system will be explained in detail with reference to FIGS. 1-4.

FIG. 1 illustrates a catheter system 100 for the extraction of a shape memory device from inside a tubular organ, in accordance with a preferred embodiment of the invention. In this regard, the catheter system 100 has two parts, which include a distal end 102 and a proximal end 104. The distal end 102 is the part of the catheter system 100 that is inserted inside the lumen of the tubular organ, where a shape memory device has been placed. The distal end 102 is a multi-lumen tube and is also referred to as multi-lumen tube 102. The proximal end 104 is the part of the catheter system 100 that is not inserted inside the lumen of the tubular organ. The proximal end 104 is used to insert an extraction device and fluid into the lumen of the tubular organ. Further, the proximal end 104 is used for moving the multi-lumen tube 102 in and out of the tubular organ. The multi-lumen tube 102 is inserted inside the lumen of the tubular organ until it reaches the point of placement of the shape memory device.

A shape memory device is made of an alloy of metals that goes through a change from one phase to another in solid state. The phase change occurs on application of pressure or a change in temperature. The two phases are martensite and austenite. In its martensitic phase, the shape memory device is soft and flexible. Further, it is easily pliable. The martensitic phase is referred to as the collapsed state when referring to shape memory devices. In its austenite phase, the shape memory device has a compact molecular structure, as compared to its martensitic phase. The austenite phase is referred to as the expanded state when referring to shape memory devices. In an embodiment of the invention, when the temperature of the shape memory device in its martensitic phase is increased, the shape memory device is converted into its austenite phase, and when the temperature in its austenite phase is lowered, the shape memory device is converted to its martensitic phase. Examples of shape memory alloys include nickel-titanium alloy (nitinol), copper-nickel-aluminum alloy, copper-zinc-aluminum alloy, and the like. In one embodiment of the invention, the shape memory device is a nitinol stent. In another embodiment of the invention, the shape memory device is a prosthetic nitinol heart valve.

In one embodiment of the invention, the shape memory device has no open or sharp edges at either end. This prevents injury to the walls of the tubular organ, thereby facilitating the removal of the shape memory device.

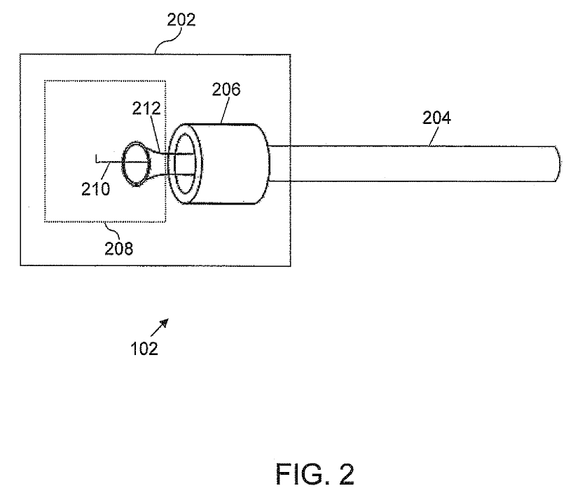

FIG. 2 illustrates the multi-lumen tube 102 of the catheter system 100 with one expandable balloon 202, in accordance with a preferred embodiment of the invention. The multi-lumen tube 102 has two or more lumens. In one embodiment of the invention, the lumens are coaxial to the axis of the multi-lumen tube 102.

In one embodiment of the invention, the multi-lumen tube 102 has a circular cross section. In case, the tubular organ is a coronary artery, the length of multi-lumen tube 102 is in the range of 100 centimeters to 120 centimeters. In case the tubular organ is a peripheral artery, the multi-lumen tube 102 has a length to accommodate placement into the treatment area near the shape memory device that is to be extracted. The diameter of the multi-lumen tube 102 facilitates the placement of the multi-lumen tube 102 into the tubular organ. In case, the tubular organ is a coronary artery, the diameter of the multi-lumen tube 102 is in the range of 0.25 centimeters to 0.50 centimeters. Further, the multi-lumen tube. 102 is made of a biocompatible polymer, such as, polyether, polyetheretherketone and polyurethane. Biocompatible polymers are well known to one of ordinary skill in the art. These materials may be utilized as single or multi-layer structures.

The multi-lumen tube 102 has an inflatable end 202 and an injectable end 204. The inflatable end 202 has an expandable balloon 206 and an extraction device 208. In one embodiment of the invention, the expandable balloon 206 has an annular cross section along its entire length. In another embodiment of the invention, the expandable balloon 206 has a circular disk-shaped cross section. The expandable balloon 206 is shown in an inflated state. The injectable end 204 is the end that is connected to the proximal end 104 of the catheter system 100 through ports. The ports are used to insert the extraction device 208, infuse fluid into the expandable balloon 206, and inject fluid inside the lumen of the tubular organ.

The expandable balloon 206 is made of a material that has suitable thermal transfer characteristics, i.e., the material is a good conductor of heat. In one embodiment of the invention, the expandable balloon 206 is made of a biocompatible conductive plastic. Examples of biocompatible conductive plastics include, but are not limited to, polytetrafluroethylene (PTFE), Dacron, and polyethylene. Biocompatible conductive plastics are well known to one of ordinary skill in the art. The expandable balloon 206 is inflated by infusing fluid into it. The infused fluid is preferably a saline solution. In one embodiment of the invention, the expandable balloon 206 is positioned to a point inside the lumen of the tubular organ, where a shape memory device in its expanded state has been deployed. The expandable balloon 206, in its inflated state, temporarily blocks the flow of blood inside the lumen of the tubular organ. The infused fluid inside the expandable balloon 206 facilitates conversion of the shape memory device from an expanded state to a collapsed state.

The extraction device 208 includes a hook 210 and a sheath 212. The hook 210 is inserted inside the lumen of the tubular organ through the sheath 212. The hook 210 is positioned such that it attaches to or is in contact with the collapsed shape memory device. The hook 210 dislodges the collapsed shape memory device, i.e., the hook 210 detaches the collapsed shape memory device from the walls of the lumen of the tubular organ. Further, the hook 210 helps to move the collapsed shape memory device into the sheath 212. In one embodiment of the invention, the hook 210 is actuated with the help of a spring-loaded push rod (not shown in the figure). This spring-loaded push rod is used to move the hook 210 back and forth inside the lumen of the tubular organ. When the spring-loaded push rod is activated, the spring is expanded, pushing the hook 210 into the lumen of the tubular organ. In its compressed state (deactivated), the spring retrieves the hook 210 out of the tubular organ, moving the collapsed shape memory device towards the sheath 212. As will be understood, a person of ordinary skill in the art can use various configurations of the hook 210, for example, a clip, a fork, or prong that will move the collapsed shape memory device towards the sheath 212.

The sheath 212 is used for transporting the shape memory device, in a collapsed state, out of the tubular organ. The sheath 212 is preferably made of a biocompatible compressible material such as polyethylene, silicon rubber, and the like. The sheath 212 has a proximal and a distal end. The shape of the sheath 212 facilitates the removal of the shape memory device from the lumen of the tubular organ. In one embodiment of the invention, the distal end of the sheath 212 has a conical shape. Its inside and outside diameters are larger than the inside and outside diameters of the proximal end of the sheath 212. The sheath 212 is folded and inserted through one of the lumens of the multi-lumen tube 102. When the sheath 212 enters into the lumen of the tubular organ, the distal end expands. In the expanded state, the diameter of the distal end of the sheath 212 is greater than the diameter of the multi-lumen tube 102. This helps it to receive the dislodged shape memory device. Further, when the sheath 212 holding the dislodged shape memory device is retrieved through one of the lumens of the multi-lumen tube 102, the distal end of the sheath 212 is folded. This facilitates the transport of the dislodged shape memory device out of the tubular organ. Further, the compressibility of the sheath 212 facilitates its movement through one of the lumens of multi-lumen tube 102.

Catheter systems often have problems with the stent becoming embedded within the sheath in which it is disposed. To overcome this problem, the sheath preferably comprises an outer polymer, preferably polyamide, layer and an inner polymer, preferably polytetrafluroethylene, layer. Other suitable polymers for the inner and outer layers include any suitable material known to those skilled in the art, including polyethylene or polyamide, respectively. Positioned between the outer and inner layers is a wire-reinforcing layer, which is preferably a braided wire. The braided reinforcing layer is preferably made of stainless steel. The use of braiding reinforcing layers can be found in U.S. Pat. No. 3,585,707 issued to Stevens on Jun. 22, 1971, U.S. Pat. No. 5,045,072 issued to Castillo et al. on Sep. 3, 1991, U.S. Pat. No. 5,254,107 issued to Soltesz on Oct. 19, 1993, and U.S. Pat. No. 6,019,778 issued to Wilson et al. on Feb. 1, 2000, all of which are hereby incorporated herein by reference.

The three layers of the sheath 212 collectively enhance the removal of the shape memory device. The layers give the sheath 212 better pushability, which is the ability to transmit a force applied by the physician at a proximal location on the sheath to the distal tip that aids in navigation across tight stenotic lesions within the vascular anatomy. The braid layer gives the sheath 212 better resistance to elongation and necking, as a result of tensile loading during sheath retraction for stent removal. The configuration of the braid layer can be changed to change system performance. This is achieved by changing the pitch of the braid, the shape of the individual braid wires, the number of braid wires, and the braid wire diameter. Additionally, coils can be incorporated similarly to the braid layer of the sheath, to enhance system flexibility. The use of coils in catheters can be found in U.S. Pat. No. 5,279,596 issued to Castaneda et al. on Jan. 18, 1994, which is hereby incorporated herein by reference.

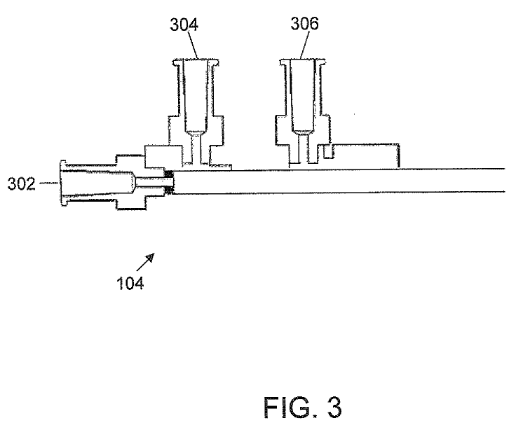

FIG. 3 illustrates the proximal end 104 of the catheter system 100, in accordance with a preferred embodiment of the invention. The proximal end 104 has three ports, hereinafter referred to as ports 302, 304 and 306. Each port is connected to at least one lumen of the multi-lumen tube 102. The ports 302, 304 and 306 are used to inject fluid and insert the extraction device 208 into the lumens of the multi-lumen tube 102. The ports 302, 304 and 306 have annular clearance, which facilitates the injection of fluid and insertion of the extraction device 208. In one embodiment of the invention, the extraction device 208 is inserted through the port 302. The fluid is injected with the help of a pump or an infusion apparatus through the port 304. In another embodiment of the invention, the fluid is injected by means of a syringe or pressure bag through the port 304. The expandable balloon 206 is inflated by infusing fluid into it through the port 306. The ports 302, 304 and 306 have one-way valves to prevent back flow of the infused fluid or bodily fluids.

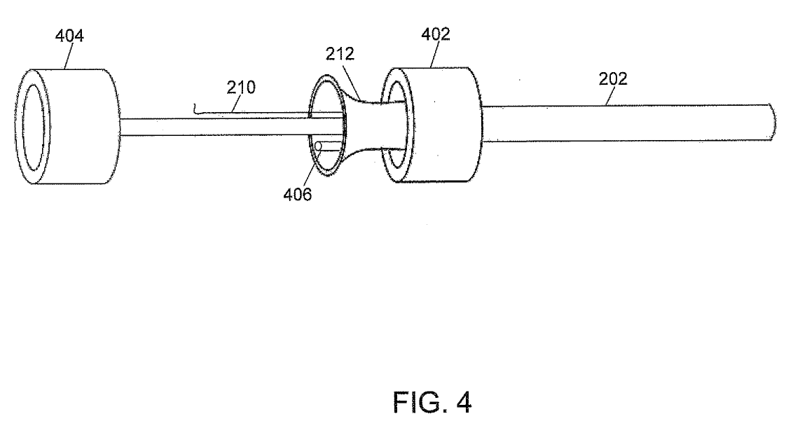

FIG. 4 illustrates the distal end 102 of the catheter system 100 with two expandable balloons, in accordance with a preferred embodiment of the invention. The multi-lumen tube 102 is inserted inside the lumen of the tubular organ, such as a coronary artery, till it reaches the point of placement of the shape memory device. The inflatable end of the multi-lumen tube 102 has two expandable balloons 402 and 404. Expandable balloons are shown in their inflated state. The expandable balloons 402 and 404 are preferably made of a material with suitable thermal transfer characteristics, i.e., the material is a good conductor of heat. In one embodiment of the invention, the expandable balloons 402 and 404 are made of a biocompatible conductive plastic. Examples of biocompatible conductive plastics include, but are not limited to, polytetrafluroethylene (PTFE) and polyethylene. The expandable balloons 402 and 404 are placed at the proximal and distal ends of the shape memory device, and are thereafter inflated with fluid, as described herein. The expandable balloons 402 and 404 block the flow of blood inside the lumen of the tubular organ, where the shape memory device is placed. Further, the expandable balloons 402 and 404 create a blocked space around the shape memory device. A cooling tube 406 infuses fluid into the blocked space and is inserted through one of the ports. The blocked space enables additional heat transfer between the shape memory device and the infused fluid. This helps in converting the shape memory device from an expanded state to a collapsed state. Once the shape memory device is collapsed, the hook 210 and the sheath 212 are employed to extract the collapsed shape memory device from the tubular organ, as described herein.

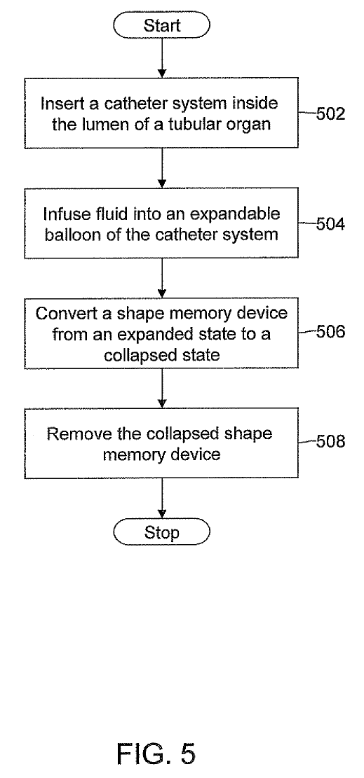

FIG. 5 is a flowchart illustrating the steps of the percutaneous extraction of a shape memory device from inside a tubular organ, in accordance with a preferred embodiment of the invention. In one embodiment of the invention, the tubular organ is an artery, preferably a coronary artery. At step 502, a catheter system is inserted inside the lumen of the tubular organ till the site of placement of the shape memory device. The shape memory device is in an expanded state, i.e., it is in the austenite phase. The catheter system is inserted into the lumen of the tubular organ through a percutaneous route, thereby making the insertion minimally invasive. At step 504, fluid is infused into an expandable balloon in the catheter system to inflate the balloon. In one embodiment of the invention, the infused fluid is a saline solution. In another embodiment of the invention, the temperature of the infused fluid is in the range of −20° Celsius to 5° Celsius.

At step 506, the infused fluid converts the shape memory device from the expanded state to a collapsed state. This is because the infused fluid reduces the temperature of the shape memory device, thereby converting it to its martensitic phase from its austenite phase. In one embodiment of the invention, the shape memory device is a nitinol stent, preferably a nitinol coronary stent. In another embodiment of the invention, the shape memory device is a prosthetic nitinol heart valve. Thereafter, at step 508, the collapsed shape memory device is removed from inside the tubular organ.

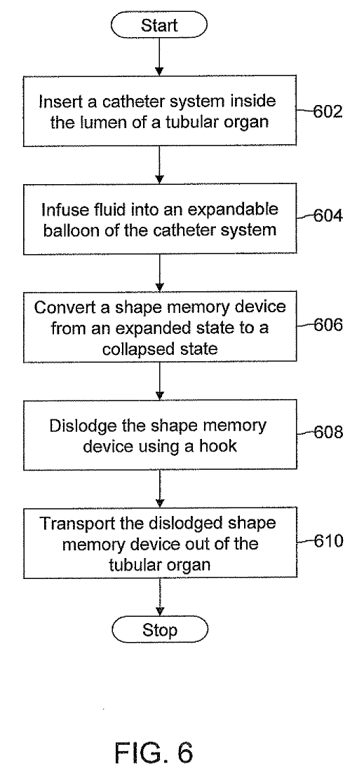

FIG. 6 is a flowchart illustrating the steps of percutaneous extraction of a shape memory device from inside a tubular organ by using an expandable balloon, in accordance with another embodiment of the invention. In one embodiment of the invention, the tubular organ is an artery, preferably a coronary artery. At step 602, a catheter system is inserted inside the lumen of the tubular organ fill the site of placement of the shape memory device. The catheter system has one expandable balloon. At step 604, fluid is infused into the expandable balloon to inflate the balloon. In one embodiment of the invention, the infused fluid is a saline solution. The temperature of the infused fluid is in the range of −20° Celsius to 50° Celsius. At step 606, the shape memory device is converted from an expanded state to a collapsed state. The temperature of the infused fluid reduces the temperature of the shape memory device, thereby converting the shape memory device to the collapsed state. At step 608, the collapsed shape memory device is dislodged from its location by using an extraction device. In one embodiment of the invention, a hook is used to dislodge the collapsed shape memory device from the walls of the tubular organ. Further, the hook moves the collapsed shape memory device into a sheath. The hook is actuated by using a spring-loaded push rod.

In one embodiment of the invention, there can be a tissue growth around the shape memory device. Hence, before dislodging the shape memory device, the tissue growth has to be removed. The tissue growth can be removed with the help of a laser device or by mechanical means employing standard, well-known ablation techniques.

Thereafter, at step 610, the dislodged and collapsed shape memory device is transported out of the tubular organ by using the sheath. The sheath holds the shape memory device in the collapsed state.

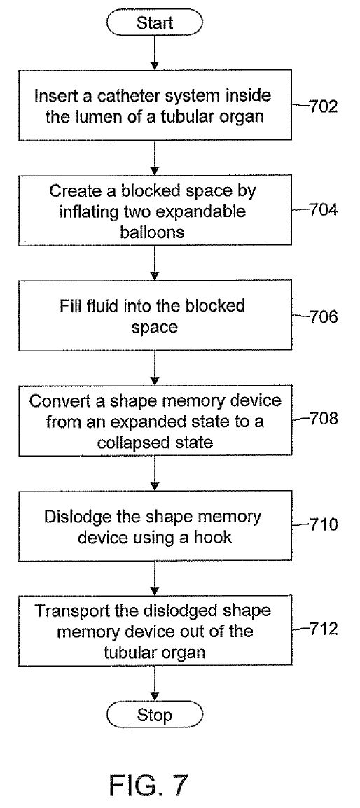

FIG. 7 is a flowchart illustrating the steps of percutaneous extraction of a shape memory device from inside a tubular organ by using two expandable balloons, in accordance with a preferred embodiment of the invention. In one embodiment of the invention, the tubular organ is an artery, preferably a coronary artery.

At step 702, a catheter system is inserted inside the lumen of the tubular organ, where an expanded shape memory device is located. At step 704, a blocked space is created around the expanded shape memory device by inflating the two expandable balloons. One expandable balloon is placed proximal and the other expandable balloon is placed distal to the expanded shape memory device. After the two balloons are inflated at step 706, fluid is filled into the blocked space between the two balloons. In one embodiment of the invention, the infused fluid is a saline solution. The temperature of the infused fluid is in the range of −20° Celsius to 5° Celsius. The fluid reduces the temperature of the shape memory device, converting it to a collapsed state from an expanded state at step 708.

At step 710, the collapsed shape memory device is dislodged from the walls of the tubular organ by using a hook. Further, the hook moves the dislodged shape memory device into a sheath. At step 712, the dislodged shape memory device is transported out of the tubular organ by using the sheath.

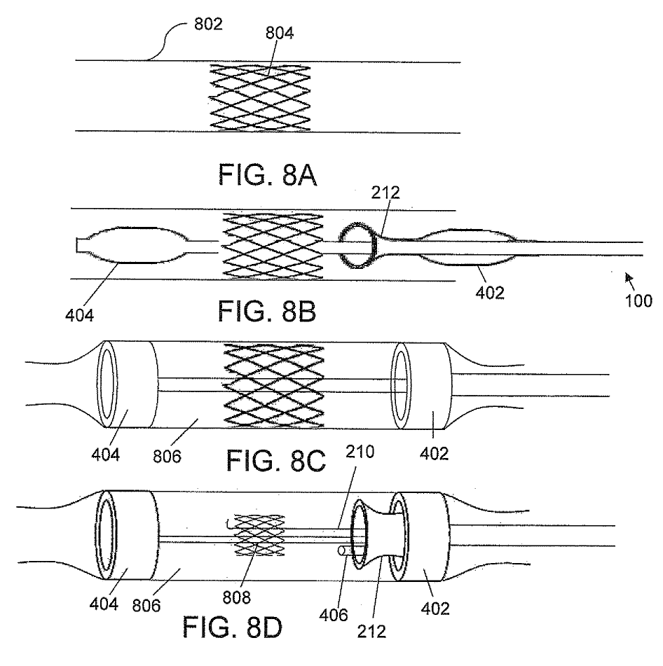

FIGS. 8A, 8B, 8C, and 8D illustrate the four stages of extraction of a shape memory device from inside the lumen of a tubular organ, according to one embodiment of the invention.

FIG. 8A shows a schematic representation of a tubular organ 802 with a shape memory device 804, according to one embodiment of the invention. The tubular organ 802 is an artery, preferably a coronary artery. The shape memory device 804 is in its expanded state, i.e., it is in its austenite phase.

FIG. 8B shows a schematic representation of the tubular organ 802 with the catheter system 100 having two expandable balloons inserted inside the lumen of the tubular organ 802, according to one embodiment of the invention. The catheter system 100 is inserted till the point of placement of the shape memory device 804. The catheter system 100 is in a deflated state, i.e., the expandable balloons 402 and 404 are in a deflated state.

FIG. 8C shows a schematic representation of the tubular organ 802 with the catheter system 100 having two expandable balloons in an inflated state inserted inside the lumen of the tubular organ 802, in accordance with a preferred embodiment of the invention. The expandable balloons 402 and 404 are in an inflated state. The expandable balloons 402 and 404 are inflated by infusing fluid into them. In one embodiment of the invention, the infused fluid is a saline solution. Inflated expandable balloons 402 and 404 create a blocked space 806 around shape memory device 804.

FIG. 8D is a schematic representation of the tubular organ 802 with the catheter system 100, along with the hook 210 and the sheath 212 inserted inside the lumen of the tubular organ, according to an embodiment of the invention. Fluid is injected into the blocked space 806 by using the cooling tube 406. The temperature of the fluid converts the shape memory device 804 from an expanded state to a collapsed state, i.e., the fluid lowers the temperature of shape memory device 804. In its collapsed state, shape memory device is referred to as a shape memory device 808. The hook 210 dislodges the shape memory device 808 from the walls of the lumen of the tubular organ 802, and moves the shape memory device 808 back into the sheath 212. The dislodged shape memory device 808 is transported out of tubular organ 802 by using the sheath 212.

In another embodiment of the present invention, the catheter system 100 may be used for deploying a shape memory device inside a tubular organ such as a coronary artery. The shape memory device, in the collapsed state, is inserted inside the lumen of the tubular organ with the help of the multi-lumen tube 102. The expandable balloon 206 blocks the flow of fluid inside the lumen of the tubular organ. The sheath 212 is used to hold and transport the shape memory device to a site, where the shape memory device is to be deployed. The hook 210 is used for positioning the shape memory device. Infused fluid assists in converting the shape memory device from the collapsed state to an expanded state, i.e., the infused fluid increases the temperature of the shape memory device. In one embodiment of the invention, the temperature of the infused fluid is 37° Celsius. In an embodiment of the invention, the collapsed shape memory device is converted to the expanded state without fluid being infused into the lumen of the tubular organ. The temperature of blood increases the temperature of the shape memory device, which converts itself to the expanded state.

The system and method described above has a number of advantages. The catheter system is minimally invasive. The simplicity of the design makes the catheter system convenient to use and cost effective.

Although the invention has been described and illustrated with a certain degree of particularity, it is understood that the present disclosure has been made only by way of example, and that numerous changes in the combination and arrangement of the parts can be resorted to by those skilled in the art, without departing from the spirit and scope of the invention, as hereinafter claimed.

We claim:

1. A catheter system for extraction of a shape memory device from inside an organ, the catheter system comprising:
a multi-lumen tube comprising an inflatable end, the inflatable end having at least one expandable balloon and an injectable end, the injectable end having multiple ports, wherein one of the ports is used to infuse fluid through one of the lumens of the multi-lumen tube into the expandable balloon; and
an extraction device, the extraction device being introduced through one of the ports into one of the lumens of the multi-lumen tube, wherein the extraction device retrieves the shape memory device by converting the shape memory device from an expanded state to a collapsed state using the infused fluid.

2. The catheter system of claim 1, wherein the extraction device comprises:
a hook, for dislodging the shape memory device from inside the lumen of the organ; and
a sheath for receiving the dislodged shape memory device.

3. The catheter system of claim 2, wherein the hook is made up of stainless steel.

4. The catheter system of claim 2, wherein the sheath is pulled to transport the shape memory device out of the organ.

5. The catheter system of claim 2, wherein the sheath is comprised of a biocompatible compressible material selected from the group consisting of polyethylene and silicon rubber.

6. The catheter system of claim 2, wherein the extraction device further comprises a spring loaded push rod, wherein the spring loaded push rod is connected to the hook, the spring loaded push rod being used to actuate the hook.

7. The catheter system of claim 1, wherein each port is joined to at least one lumen of the multi-lumen tube.

8. The catheter system of claim 1, wherein the expandable balloon is comprised of biocompatible conductive plastic selected from the group consisting of polytetrafluroethylene (PTFE) and polyethylene.

9. The catheter system of claim 1, wherein the shape memory device is made up of a material selected from a group comprising Ni—Ti alloy, Cu—Al—Ni alloy, and Cu—Zn—Al alloy.

10. The catheter system of claim 1, wherein the infused fluid is a cold saline solution.

11. The catheter system of claim 1, wherein the shape memory device is a stent or a heart valve.

12. A catheter system for extraction of a nitinol stent from inside a blood vessel, the catheter system comprising:
a multi-lumen tube for insertion into the blood vessel comprising an inflatable end, the inflatable end having at least one expandable balloon and an injectable end, the injectable end having multiple ports, wherein one of the ports is used to infuse fluid through one of the lumens of the multi-lumen tube, the temperature of the infused fluid helping to convert the nitinol stent from austenite state to martensitic state; and
an extraction device, the extraction device being introduced through one of the polls into one of the lumens of the multi-lumen tube, the extraction device comprising a hook, the hook being used to dislodge the nitinol stent from inside the lumen of the blood vessel, wherein the hook is attached to a spring loaded push rod, the spring loaded push rod being used to actuate the hook and a sheath, the sheath being used to transport the dislodged nitinol stent out of the tubular organ.

13. A method for percutaneous extraction of a shape memory device from inside an organ using a catheter system, the catheter system comprising a multi-lumen tube and an extraction device, the method comprising:
inserting the catheter system into the organ till the catheter device reaches the point of placement of the shape memory device;
infusing fluid into the catheter system through one of the lumens of the multi-lumen tube for converting the shape memory device from an expanded state to a collapsed state using the infused fluid; and
removing the shape memory device in the collapsed state from the organ.

14. The method of claim 13, wherein the insertion of the catheter system into the organ is performed percutaneously.

15. The method of claim 13, wherein the extraction device further comprises a hook and a sheath and wherein removing the shape memory device comprises:
dislodging the shape memory device using the hook of the extraction device; and
transporting the dislodged shape memory device out of the organ using the sheath of the extraction device.
